(19) United States
(12) Patent Application Publication
(10) Pub. No.: US 2022/0057136 A1
(43) Pub. Date: Feb. 24, 2022

(54) Cooling Unit

(71) Applicant: Brian S. KIM, Rancho Palos Verdes, CA (US)

(72) Inventor: Brian S. KIM, Rancho Palos Verdes, CA (US)

(21) Appl. No.: 16/996,174

(22) Filed: Aug. 18, 2020

(51) Int. Cl. F25D 27/00; F21V 23/00; F21S 4/28; F21V 33/00; A61L 2/10

(52) U.S. Cl. CPC F25D 27/00; F21V 23/001; F21Y 2115/10; F21V 33/0064; A61L 2/10; F21S 4/28

(57) ABSTRACT

A cooling unit has an internal chamber having a linear lighting source (an in-line lighting source) that has a row of light sources. The row of light sources are has at least one visible light section and at least one ultraviolet light section. The visible light sections and the ultraviolet light sections are all aligned in a single line and the visible light section and the ultraviolet light section are alternatingly placed in the line so a visible light section is adjacent to a ultraviolet light section. The visible light section has one or more visible light emitting LEDs and the ultraviolet light section has one or more ultraviolet light emitting LEDs. Each of the visible light emitting LEDs and the ultraviolet light emitting LEDs are all aligned in the line, as well as the visible light sections and the ultraviolet light sections.

### SUMMARY

A cooling unit with an in-line light-emitting diodes (LEDs) is presented herein. The in-line light-emitting diodes have both visible light-emitting diodes and ultraviolet light emitting light-emitting diodes (LEDs) aligned in a single row (or in a single line).

There are cooling units, including but not limited to, refrigerators or freezers, with both a visible light source and a ultraviolet light source, such as the ones disclosed in U.S. Pat. No. 8,387,405B2, by Johnson, and the one disclosed in U.S. Patent Publication 2013/0104579, by Zhou, both publications are incorporated herein by reference. However, there has not been a cooling unit with an in-line light-emitting diodes (LEDs) incorporating both the visible light-emitting diodes and ultraviolet light emitting light-emitting diodes aligned in a single line, such that a group of one or more light-emitting diodes of the visible light and a group of one or more light-emitting diodes of the ultraviolet light, wherein each group is aligned in a line and each group is alternatingly positioned as shown in FIGS. 1-3.

One of the advantages of cooling unit with an in-line light-emitting diodes (LEDs), as described herein, is the space saving achieved because both the visible light source and the ultraviolet light source are in a single strip and therefore could be placed at any location, especially on a narrow strip—such as on the frontside of a thin shelf used in a refrigerator. Moreover, there is no need to have a large visible light producing light bulb in the cooling unit chamber. Also, each internal compartment or internal space, such as in-between space between two shelves or inside of a drawer of the refrigerator, can now be directly illuminated by visible light and directly treated with ultraviolet light for sanitation purpose, effecting ultraviolet light induced disinfection. Thus, each compartment or each drawer can have its own visible light source and disinfecting ultraviolet light source from a compact in-line (that is, in a linear arrangement) light-emitting diodes that is simple, thin and space saving, as well as made into any length.

A cooling unit, including but not limited to, a refrigerator and freezer, has an internal chamber with a door. The chamber has a linear lighting source (an in-line lighting source) that has a row of light sources. The row of light sources has at least one visible light section and at least one ultraviolet light section. The visible light sections and the ultraviolet light sections are all aligned in a single line and each of the visible light section and each of the ultraviolet light section is alternatingly placed in the line so a visible light section is adjacent to an ultraviolet light section and vice versa.

The visible light section has one or more visible light emitting light-emitting diodes (LEDs) and the ultraviolet light section has one or more ultraviolet light emitting light-emitting diodes (LEDs). Each of the visible light emitting LEDs and the ultraviolet light emitting LEDs are all aligned in the line, as well as the visible light sections and the ultraviolet light sections are aligned in the line.

The linear lighting source also has two endcaps, a first endcap located at one end of the linear lighting source and the second endcap located at the opposite end of the linear lighting source. The linear lighting source has a set of cables running through (or attached to) an endcap to deliver power to the LEDs. The linear lighting source may have a cover that allows the lights from the light-emitting diode to pass through and to protect the LEDs.

Because the linear lighting source is a thin and narrow line of LEDs, generating both the visible light and the ultraviolet light, it is simple, narrow and easily applied to tight and narrow spaces. For example, the linear lighting source may be placed under each shelf of the cooling unit. In addition, the linear lighting source may be located on the front end of each shelf. Because the linear lighting source has both the visible light source and the ultraviolet light source in a thin line, the linear lighting source may also be placed on each of the edges of the shelves, as well as on each of the frontside of the shelves. However, to expose the most area under the visible light and the ultraviolet light, it is preferable that the linear lighting source is located under each shelf, as well as on the upper surface of the chamber and on the lower surface of the chamber (or on the edge of the lower surface, near the door, or on the lower rear end of the chamber). It shall be noted that the linear lighting source has numerous LEDs spaced apart and thus the linear lighting source may be flexible. The linear lighting source may be thus bent into a circular shape or elliptical shape or a “U” shape to be placed under a shelf of the refrigerator to sanitize the most surface area under it.

Although the present invention is briefly summarized, a better understanding of the invention can be obtained by the following drawings, detailed description and appended claims.

### BRIEF DESCRIPTION OF THE DRAWINGS

These and other features, aspects and advantages of the cooling unit with an in-line light-emitting diodes (LEDs) will become better understood with reference to the accompanying drawings, wherein:

FIG. 1: a variation of the linear lighting source (an in-line lighting source),

FIG. 2: a variation of the linear lighting source,

FIG. 3: a variation of the linear lighting source,

FIG. 4: the cooling unit showing the linear lighting source mounted on the upper surface of the cooling unit and on each of the shelves,

FIG. 5: the cooling unit showing the linear lighting source mounted on the upper surface of the cooling unit and on the right side of the chamber,

FIG. 6: the cooling unit showing the linear lighting source mounted on the upper surface of the cooling unit and on the left side of the chamber,

FIG. 7: the cooling unit showing the linear lighting source mounted on the upper surface of the cooling unit and near the door of the chamber,

FIG. 8: the cooling unit showing the linear lighting source mounted on the lower surface of the cooling unit, near the door of the cooling unit,

FIG. 9: a variation of the linear lighting source,

FIG. 10: a side elevation view of the linear lighting source shown on FIG. 9,

FIG. 11: an isometric view of the linear lighting source shown on FIG. 9, and

FIG. 12: a simplified electric schematic of the cooling unit showing the connection to the linear lighting source.

### DETAILED DESCRIPTION EMBODIMENTS OF THE INVENTION

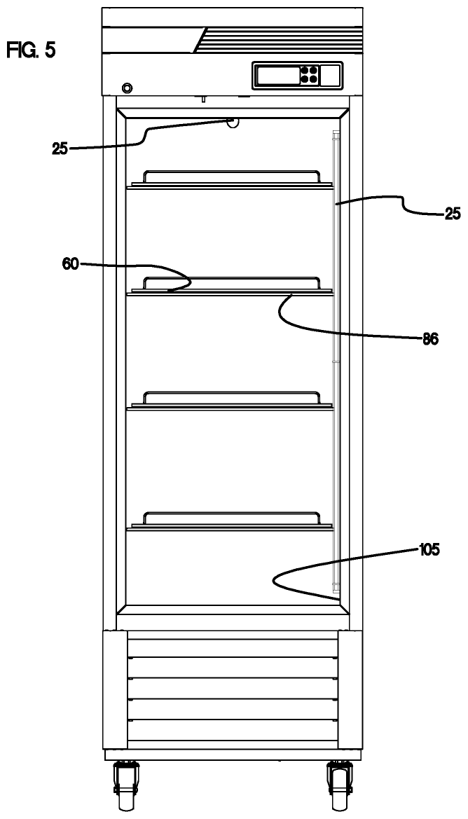

A cooling unit 10 (see FIGS. 4-8) with an in-line light-emitting diodes (LEDs) comprising of both a plurality of visible light emitting light-emitting diodes (LEDs) 15 and a plurality of ultraviolet light emitting light-emitting diodes (LEDs) 20. Each of the plurality of visible light emitting light-emitting diodes 15 and each of the plurality of ultraviolet light emitting light-emitting diodes 20 are aligned in a single line. The cooling unit 10 is described in detail as shown on the Figs.

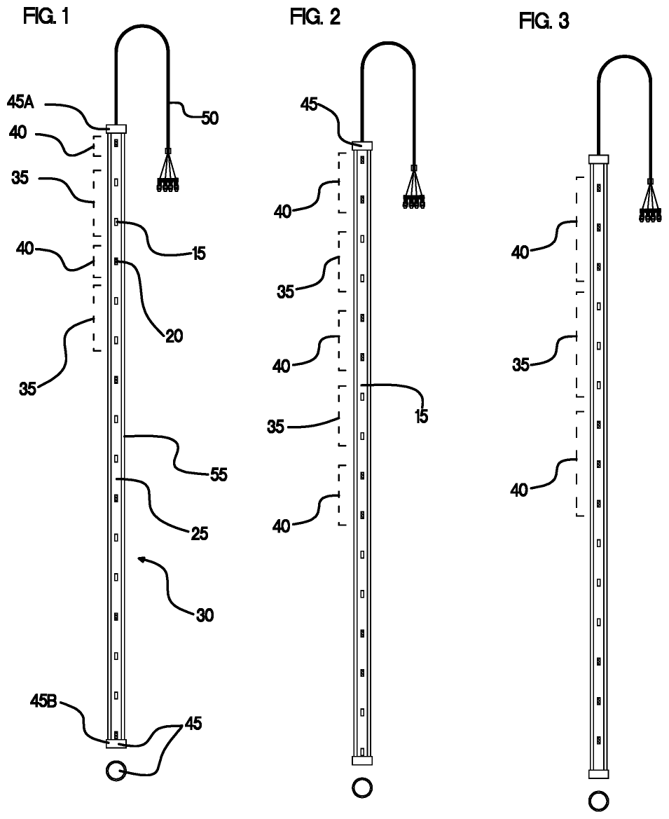

FIG. 1 shows a linear lighting source 25 (an in-line lighting source) that is formed by a row of light sources 30 which are made of at least one visible light section 35 and of at least one ultraviolet light section 40. The visible light sections 35 and the ultraviolet light sections 40 are all aligned in a single line and the visible light sections 35 and the ultraviolet light sections 40 are alternatingly placed in the line, so a visible light section 35 is placed adjacent to a ultraviolet light section 40 and vice versa.

The visible light section 35 has one or more visible light emitting light-emitting diodes (LEDs) 15 and the ultraviolet light section 40 has one or more ultraviolet light emitting light-emitting diode (LEDs) 20. Each of the visible light emitting light-emitting diodes 15 and each of the ultraviolet light emitting light-emitting diodes 20 are all aligned in a line, as well as the visible light sections 35 and the ultraviolet light sections 40 are also all aligned in the line.

The linear lighting source 25 also has two endcaps 45, a first endcap 45A located at one end of the linear lighting source 25 and the second endcap 45B located at the opposite ends of the linear lighting source 25. The linear lighting source 25 has a cable 50 running through or attached to an endcap 45 to receive power. FIG. 1 also shows an end view of the linear lighting source 25, showing an endcap 45.

The linear lighting source 25 may have a cover 55 that allows the lights from the light-emitting diodes 15, 20 to pass through (that is, transparent) and protects the LEDs 15, 20. The cover 55 may be rigid or flexible. A clear plastic tube may be used for a flexible cover 55.

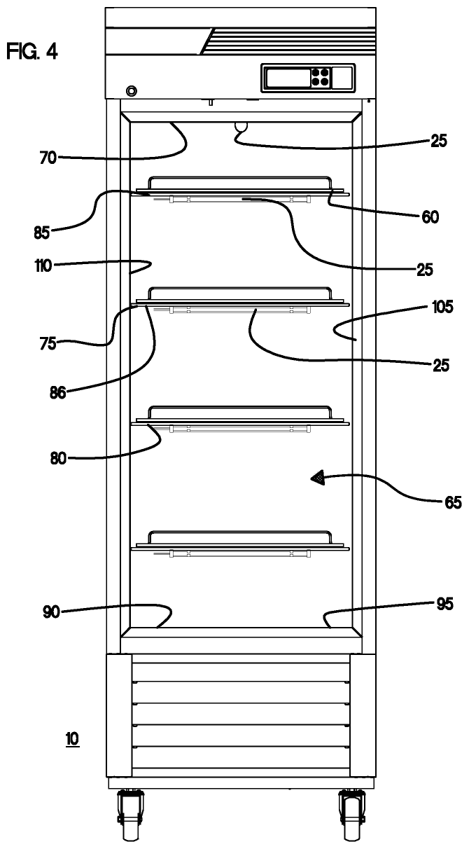

FIG. 2 shows a linear lighting source 25 (an in-line lighting source) with each of the visible light sections 35 having at least two visible light emitting light-emitting diodes 15 and each of the ultraviolet light section 40 having at least two ultraviolet light emitting light-emitting diodes 20. Each of the visible light emitting light-emitting diodes 15 and each of the ultraviolet light emitting light-emitting diodes 20 are all aligned in the line.

FIG. 3 shows a linear lighting source 25 (an in-line lighting source) with each of the visible light sections 35 having at least three visible light emitting light-emitting diodes 15 and each of the ultraviolet light sections 40 having at least three ultraviolet light emitting light-emitting diodes 20. Each of the visible light emitting light-emitting diodes 15 and each of the ultraviolet light emitting light-emitting diodes 20 are all aligned in the line.

FIGS. 1-3 are shown to illustrate that a linear lighting source 25 (an in-line lighting source) may be designed with each of the visible light sections 35 to have one or more visible light emitting light-emitting diodes 15 and each of the ultraviolet light sections 40 to have one or more ultraviolet light emitting light-emitting diodes 20, but having different number of ultraviolet light emitting light-emitting diodes 20 in the ultraviolet light section 40 from the number of the visible light emitting light-emitting diodes 15 in the visible light section 35. With whatever number of LEDs 15, 20 in each visible light section 35 or in each ultraviolet light section 40, each of the visible light emitting light-emitting diodes 15 and each of the ultraviolet light emitting light-emitting diodes 20 are all aligned in the line.

FIG. 4 shows the cooling unit 10 with shelves 60 in its chamber 65. The cooling unit 10 has a linear lighting source 25 attached to the upper surface 70 of the chamber 65. In addition, because the linear lighting source 25 is a thin and narrow line of LEDs 15, 20, illuminating with both the visible light and the ultraviolet light, it is simple, narrow and easily applied to tight spaces. As an example, as shown in FIG. 4, the cooling unit 10 has a linear lighting source 25 attached on the front end 75 of each shelf 60. In addition, because the linear lighting source 25 has both the visible light source 35 and the ultraviolet light source 40 in a thin line, the linear lighting source 25 may be placed on the edges 80 of the shelves 60, as well as the narrow frontside 85 of the shelves 60.

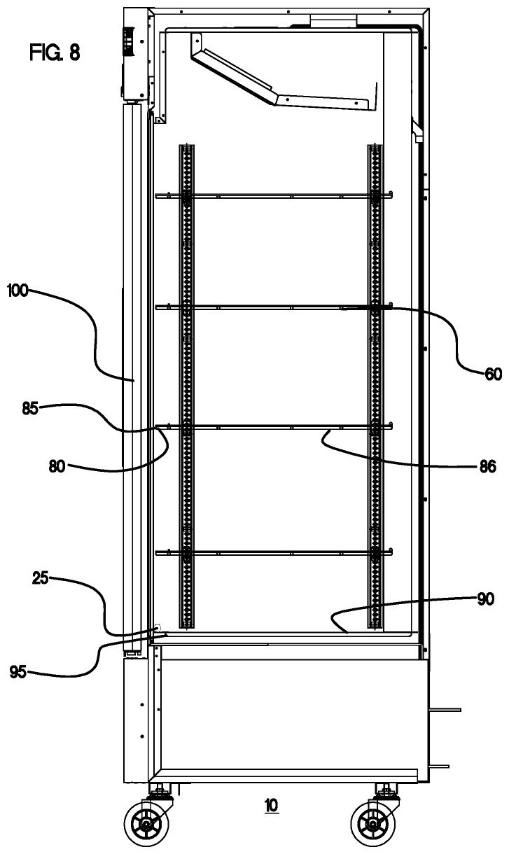

However, to expose the most area under the visible light and the ultraviolet light, it is preferable that the linear lighting source 25 is located underneath 86 each shelf 60, as well as on the upper surface 70 of the chamber 65 and on the lower surface 90 of the chamber 65. The linear lighting source 25 may be place also on the edge of the lower surface 95 (as shown in FIG. 8, near the door 100, or on the lower rear end of the chamber). Also, the linear lighting source 25 may be placed under each shelf 60 of the cooling unit 10.

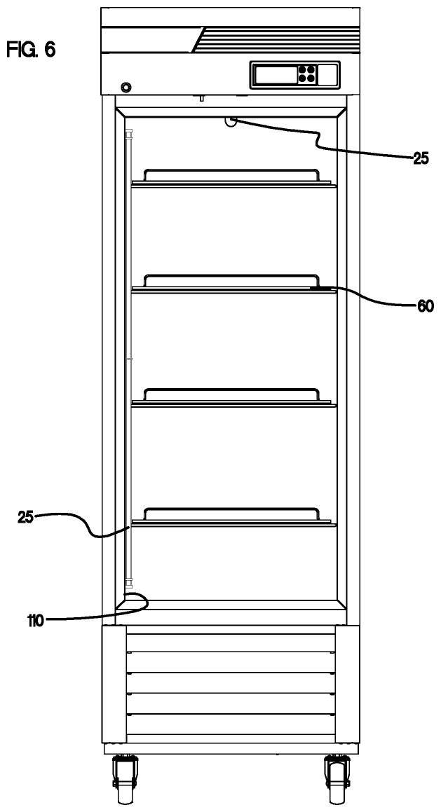

FIG. 5 shows the cooling unit 10 with shelves 60 in its chamber 65. The cooling unit 10 has a linear lighting source 25 attached to the upper surface 70 of the chamber 65 and a linear lighting source 25 attached to the right side 105 of the chamber 65.

FIG. 6 shows the cooling unit 10 with shelves 60 in its chamber 65. The cooling unit 10 has a linear lighting source 25 attached to the left side 110 of the chamber 65.

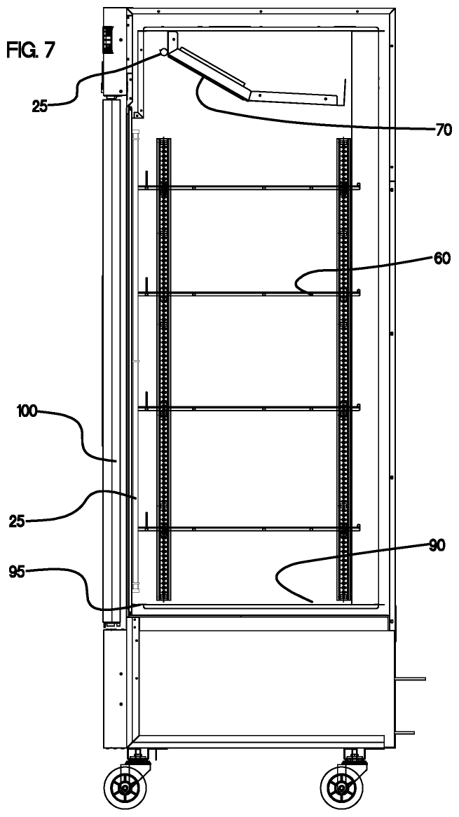

FIG. 7 shows the cooling unit 10 with shelves 60 in its chamber 65. The cooling unit 10 has a linear lighting source 25 attached inside of the chamber 65, near the door 100, on the right side 105.

FIG. 8 shows the cooling unit 10 with shelves 60 in its chamber 65. The cooling unit 10 has a linear lighting source 25 attached inside of the chamber 65, near the door 100, on the right side 105, on the bottom surface of the chamber 65.

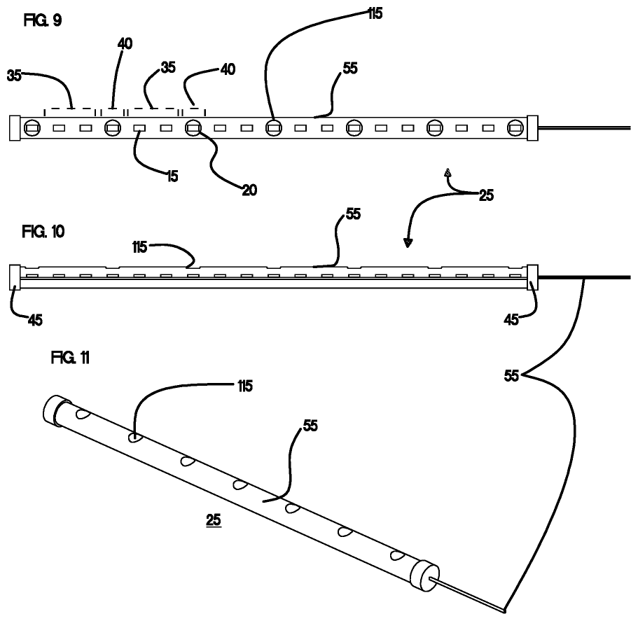

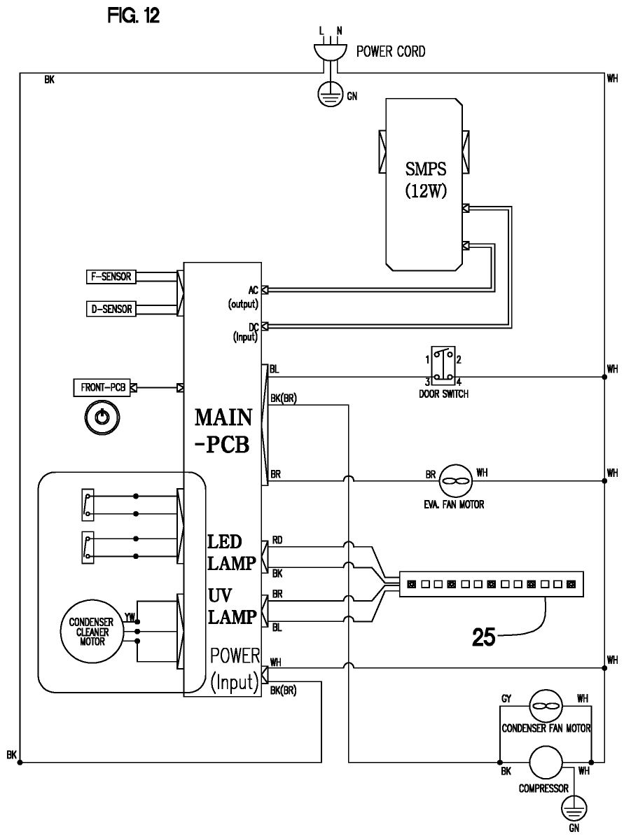

FIGS. 9-11 show a variation of the linear lighting source 25 with the visible light sections 35 having two visible light emitting light-emitting diodes 15 and each of the ultraviolet light section 40 having one ultraviolet light emitting light-emitting diode 20. Each of the visible light emitting light-emitting diodes 15 and each of the ultraviolet light emitting light-emitting diodes 20 are all aligned in a line.

The linear lighting source 25 also has the cover 55 that is substantially transparent or clear, allowing light from LEDs to pass through and the linear lighting source 25 has two end caps 45, each placed at the opposite ends of the linear lighting source 25. The cover 55 also has a hole 115 directly over at least one of the ultraviolet light emitting light-emitting diode 20 so the light from the ultraviolet light emitting light-emitting diode 20 passes through the hole 115.

FIG. 12 is a simplified electric schematic of the cooling unit 10 showing the connection to a linear lighting source 25. As seen by the electric schematic, the main PCB can control each visible light LED and each Ultraviolet light LED or each group thereof. As an example, the ultraviolet light may be turned on and the visible light may be turned off when the door is closed, the ultraviolet light may be turned off and the visible light may be turned on when the door is open, or both the ultraviolet light and the visible light continuously stay on when the door is closed. The power supplied to the LEDs may be adjusted to control the illumination strength, to be adjusted adjusting according to the ambient lighting.

While the description, drawings and references have presented, shown and described with reference to different embodiments thereof, it will be appreciated by those skilled in the art that variations in form, detail, compositions and operation may be made without departing from the spirit and scope of the disclosure.

What is claimed is:

1. A cooling unit comprising a chamber with a door, wherein the chamber has a linear lighting source, wherein the linear lighting source comprises of a row of light sources, wherein the row of light sources comprises of a visible light section and of a ultraviolet light section aligned in a line, wherein the visible light section and the ultraviolet light section are alternatingly placed in the line, wherein the visible light section comprises of a visible light emitting light-emitting diode and the ultraviolet light section comprises of a ultraviolet light emitting light-emitting diode, and wherein the visible light emitting light-emitting diodes and the ultraviolet light emitting light-emitting diodes are all aligned in the line.

2. The cooling unit of claim 1 wherein the linear lighting source further comprises of a cable receiving power.

3. The cooling unit of claim 2 wherein the linear lighting source further comprises of a first endcap located at one end of the linear lighting source and the second endcap located at the opposite ends of the linear lighting source.

4. The cooling unit of claim 3 wherein the linear lighting source further comprises of a cover that allows light from the light-emitting diode to pass through.

5. The cooling unit of claim 3 further comprising a shelf, wherein the linear lighting source is located under the shelf.

6. The cooling unit of claim 3 further comprising a shelf, wherein the linear lighting source is located on the front end of the shelf.

7. The cooling unit of claim 3 wherein the linear lighting source is located on a upper surface of the cooling unit.

8. The cooling unit of claim 2 wherein the linear lighting source further comprises of a cover that allows light from the light-emitting diode to pass through, wherein the cover has a hole directly over the ultraviolet light emitting light-emitting diode.

9. The cooling unit of claim 1 wherein each of the visible light section comprises of at least two visible light emitting light-emitting diodes and each of the ultraviolet light section comprises of at least two ultraviolet light emitting light-emitting diodes, and wherein each of the visible light emitting light-emitting diodes and each of the ultraviolet light emitting light-emitting diodes are all aligned in the line.

10. The cooling unit of claim 9 wherein the linear lighting source further comprises of a first endcap located at one end of the linear lighting source and the second endcap located at the opposite ends of the linear lighting source.

11. The cooling unit of claim 9 wherein the linear lighting source further comprises of a cover that allows light from the light-emitting diode to pass through, wherein the cover has a hole directly over at least one of the ultraviolet light emitting light-emitting diode.

12. The cooling unit of claim 9 further comprising a shelf, wherein the linear lighting source is located under the shelf.

13. The cooling unit of claim 9 further comprising a shelf, wherein the linear lighting source is located on the front end of the shelf.

14. The cooling unit of claim 1 wherein each of the visible light section comprises of at least three visible light emitting light-emitting diodes and each of the ultraviolet light section comprises of at least three ultraviolet light emitting light-emitting diodes, and wherein each of the visible light emitting light-emitting diodes and each of the ultraviolet light emitting light-emitting diodes are also aligned in the line.

15. The cooling unit of claim 14 wherein the linear lighting source further comprises of a first endcap located at one end of the linear lighting source and the second endcap located at the opposite ends of the linear lighting source.

16. The cooling unit of claim 14 wherein the linear lighting source further comprises of a cover that allows light from the light-emitting diode to pass through, wherein the cover has a hole directly over at least one of the ultraviolet light emitting light-emitting diode.

17. The cooling unit of claim 14 further comprising a shelf, wherein the linear lighting source is located under the shelf.

18. The cooling unit of claim 1 wherein each of the visible light sections comprises of one or more visible light emitting light-emitting diodes and each of the ultraviolet light sections comprises of one or more ultraviolet light emitting light-emitting diodes, wherein the number of ultraviolet light emitting light-emitting diodes in the ultraviolet light section is different from the number of the visible light emitting light-emitting diodes in the visible light section, and wherein each of the visible light emitting light-emitting diodes and each of the ultraviolet light emitting light-emitting diodes are all aligned in the line.

19. The cooling unit of claim 18 wherein the linear lighting source further comprises of a cover that allows light from the light-emitting diode to pass through, wherein the cover has a hole directly over at least one of the ultraviolet light emitting light-emitting diode.

20. The cooling unit of claim 18 further comprising a shelf, wherein the linear lighting source is located on the front end of the shelf.
